(19) United States
(12) Patent Application Publication
(10) Pub. No.: US 2005/0273112 A1
(43) Pub. Date: Dec. 8, 2005

(54) Three-dimensional osteotomy device and method for treating bone deformities

(76) Inventor: Michael G. McNamara, Anchorage, AK (US)

Correspondence Address: BROMBERG & SUNSTEIN LLP, 125 SUMMER STREET, BOSTON, MA, 02110-1618, US

(21) Appl. No.: 11/132,718

(22) Filed: May 19, 2005

Related U.S. Application Data
(60) Provisional application No. 60/574,418, filed on May 26, 2004.

(51) Int. Cl. A61B 17/56

(52) U.S. Cl. 606/87

(57) ABSTRACT

An osteotomy device for correcting angulation, rotation, and length deformities in bones is disclosed along with a method for use of the device. A deformity of a bone can occur when there is a mal-union of first and second section of the bone or as the result of congenital mal-alignment. The osteotomy device has a body having first end and a second end and a slot between the first and second ends. The slot is designed for receiving a blade for making cuts in the bone. The slot includes a proximal end proximate to the first end of the body and a distal end that is proximate to the second end of the body. The body also includes a plurality of apertures for receiving guide pins there through for securing the body to the bone. The body can be rotated about a guide pin for making a plurality of cuts and then can be secured to the bone by using a second guide pin through a second aperture. When the body is coupled to the bone by two guide pins the cutting blade can be placed into the slot for making a secure and precise bone cut.

### CROSS REFERENCE TO RELATED APPLICATIONS

This application claims priority from U.S. provisional patent application having Ser. No. 60/574,418 entitled “Three Dimensional Osteotomy Device and Method for Treating Mal-unions” that was filed on May 26, 2004 and which is incorporated herein by reference in its entirety.

### TECHNICAL FIELD AND BACKGROUND ART

The present invention relates to a method and guide for correcting deformities such as angulation, rotation, and length abnormalities in bones including mal-unions. A mal-union occurs when a bone that is composed of two sections heals in an abnormal position such that there may be an axial deviation, mismatched length or rotational deformity. Axial deformities can occur with a poorly set fracture or broken bone. Such mal-unions, when occurring in the leg, can cause secondary degenerative joint disease because of continued weight bearing in an abnormal position. Rotational mal-unions also occur and are usually those of external rotation. Internal rotational deformities may cause more serious problems than external rotational deformities, but are less common. Most external rotational and lateral axis deformities are associated with late diagnoses, neglected treatment, or the improper positioning during the application of a cast or splint.

In the prior art there are many different methods for individually correcting either rotational or length deformities of a bone. Similarly there are techniques that are available for correcting angulation. For example, U.S. Pat. No. 4,433,681 to Camparetto teaches a bone elongation and shortening method. In this methodology, bones are cut in parallel curvilinear cuts and the bones are shortened or lengthened. The patent further teaches the use of a ribbon shaped blade or blade guide for performing the methodology. The Camparetto patent does not teach a methodology that allows for elongation/shortening, and/or correction of angulation, and/or correction of rotation.

Similarly, U.S. Pat. No. 4,632,102 to Camparetto teaches a bone wedge osteotomy method. In this patent, a wedge guide of specific angular dimension allows the excision of a precise bone wedge by the alternative use of a crescent and planar saw. The patent further teaches a guide for the crescent and planar saws. A crescent cut is made through a portion of the bone so as not to separate the bone into two portions. A planar cut is then made at the other side of the bone to the crescent cut so that the bone separates into two pieces. A second planar cut may be made toward the crescent cut forming a crescent shaped-bone section. After removal of the bone section, the bones can be repositioned to correct for the angular deformity. The methodology does not teach how to correct for elongation and for rotational deformities using the guide.

### SUMMARY OF THE INVENTION

In a first embodiment of the invention there is provided an osteotomy device for correcting angulation, rotation, and length deformities in bones. A deformity of a bone can occur when there is a mal-union of first and second section of the bone or as the result of congenital mal-alignment. The osteotomy device has a body having first end and a second end and a slot between the first and second ends. The slot is designed for receiving a blade for making cuts in the bone. The slot includes a proximal end proximate to the first end of the body and a distal end that is proximate to the second end of the body. The body also includes a plurality of apertures for receiving guide pins there through for securing the body to the bone. The body can be rotated about a guide pin for making a plurality of cuts and then can be secured to the bone by using a second guide pin through a second aperture. When the body is coupled to the bone by two guide pins the cutting blade can be placed into the slot for making a secure and precise bone cut. At least one aperture of the body is present between the first end of the body and the proximal end of the slot. In other embodiments, at least one aperture is present between the second end of the body and the distal end of the slot. The body may include a plurality of apertures between the first end of the body and the proximal end of the slot. In other embodiments of the invention, the body is symmetric wherein there are an equal number of apertures on either side of the slot. The body may also include a slot that cuts through an axis that extends through the at an angle with respect to a plane that includes the axis. In yet another embodiment, the body may include a plurality of slots, wherein each slot is positioned at a different angle with respect to the plane that includes the axis that extends through the body (0 degrees, 15 degrees, −15 degrees, 30 degrees etc.)

The methodology for correcting the deformity in the bone is performed by defining a first axis along the center of a first section of the bone that has the deformity. A second axis is defined along the center of second section of the bone that intersects with the first axis. The intersection point is the center of the deformity. A first guide pin is used for securing the jig at the intersection of the first and second axes. A second guide pin may be used for securing the jig at two points prior to making the first cut. The first cut is made along the first axis on the second section of bone using the jig to guide a blade. The blade is placed into the slot during the cutting of the bone. The jig can then be rotated about the first guide pin. A second cut is then made along the second axis in the second section of bone using the jig to guide the blade. A third cut transverse to the second cut is made such that a substantially triangular wedge of bone may be removed. A fourth cut that fully separates the first section and the second section of the bone is then made. The substantially triangular wedge of bone is removed and the first section and the second section of the bone are repositioned such that the first and second axes are substantially aligned. Thus, the angular deformity is corrected. Since the bone is cut into two separate sections and the bone sections once aligned overlap, the bone can be lengthened by moving the bone sections relative to one another. Preferably, the bone is only extendible to points at which the bone sections partially overlap. Angular deformities may also be corrected in a second plane perpendicular to the first plane in which the major angular deformity was corrected. Thus, angular correction of a lesser angle of deformity can be made by pivoting one of the bone sections on the other around an axis in the second plane.

In another embodiment of the osteotomy device, the device includes three jig pieces. The first jig has a first end and a second end with a longitudinal slot substantially parallel to an X-axis between the first end and the second end. The first jig also including a cutout wherein at least one edge of the cutout is substantially perpendicular to the longitudinal slot. Additionally, the first jig has an aperture defining a pivot point when a pin is received through the aperture. The pivot point allows the first jig to be rotatably mounted to the bone.

The second jig is shaped to fit within the longitudinal slot of the first jig and has a slot configured to receive a saw blade. The third jig has a first section and a perpendicular second section substantially parallel to a Y-axis. The first section is configured to fit within the longitudinal slot of the first jig and the second section has a slot configured to fit a saw blade. The first and second jigs are used together to make, the first and second cuts. The first and third jigs are used in combination to make the third and fourth cuts.

During the procedure for correcting the bone deformity, the bone wedge that is removed may be used as a bone graft to fill any voids left with the bone upon realignment of the first and second bone sections. After the bone sections have been realigned, a surgeon may attach a plate, such as a locking compression plate, to the first and second sections. In other embodiments the plate may be a non-locking plate. The plate may also have a component or lip to stabilize the bone graft as opposed to locking screws that buttress the bone graft.

In order to correct for rotational deformities in the bone, a jig that has a slot that is at an angle with respect to a plane that includes the first pin is used. The jig is positioned so that the jig with the angled slot is positioned on the first pin and a cut is made along the second axis on the second section of bone. This is at the same location as the second cut that was previously described. Both the second cut and the cut with the angled slot go through the entire bone. The two cuts create a bone wedge that can be removed. After the osteotomy is complete and the bone is in two sections, the bone sections can be rotated through an arc where the bone wedge was removed so as to correct for the rotational deformity.

### BRIEF DESCRIPTION OF THE DRAWINGS

The foregoing features of the invention will be more readily understood by reference to the following detailed description, taken with reference to the accompanying drawings, in which:

FIGS. 1A-1D show different views of an embodiment of a jig for osteotomies;

FIG. 1A shows a side view of the jig which has a slot for guiding a blade where the slot is at zero degrees relative to a longitudinal plane;

FIG. 1B shows a side view of the jig, which has a slot for guiding a blade where the slot is at five degrees relative to a longitudinal plane;

FIG. 1C shows a top view of an embodiment of the jig wherein the jig has a plurality of slots;

FIG. 1D show a top view of another embodiment of the jig wherein the jig includes a plurality of slots;

FIG. 2A shows a second embodiment of the jig, wherein the jig includes a base and a pivoting slotted member;

FIG. 2B shows an embodiment of the jig of FIG. 2A wherein the pivoting slotted member is removed and a slotted planar member is attached;

FIGS. 3A-3C2 shows the three components for a third embodiment of the jig;

FIG. 3A shows the pivoting base;

FIG. 3B1 shows a top view and FIG. 3B2 shows an end view of a jig element having a longitudinal slot that fits into the base;

FIG. 3C1 shows a top view and FIG. 3C2 shows an end view of a jig element having a slot that is perpendicular to the longitudinal axis that fits into the base;

FIG. 4 is a side view of a bone having a deformity along with denoted cut lines;

FIG. 5 is a side view of a bone having a deformity along with the denoted cut lines including a cut line for rotational correction;

FIG. 6 is a photograph of a radius mal-union that is short, angulated and rotated;

FIG. 7 shows a first position of the jig of FIG. 1 during correction of a bone deformity;

FIG. 8A shows a perspective view of a second position of the jig;

FIG. 8B shows a top view of a second position of the jig;

FIG. 8C shows a perspective view of a second position of the jig having an angled slot for rotational correction;

FIGS. 9A-B show a cross-sectional view and correction of a bone in a counter-clockwise rotational direction;

FIG. 9A shows the wedge that is formed after two cuts are made in the bone;

FIG. 9B shows the re-assembled bone pieces with an arrow indicating the counter-clockwise rotation of one of the bone pieces;

FIGS. 10A-C are cross-sectional views of a bone wherein the bone is being corrected in clockwise rotational direction;

FIG. 10A shows a first cut of the bone;

FIG. 10B shows a second cut that forms a bone wedge;

FIG. 10C shows the two bone pieces realigned after the bone wedge has been removed;

FIG. 11 shows the positions of the third and fourth cuts that complete the osteotomy and create a removable bone wedge;

FIG. 12A shows the two bone pieces that are formed during the osteotomy immediately after the bone wedge is removed;

FIG. 12B shows the two bone pieces after the center axes have been re-aligned;

FIG. 13 shows another view of the two axes re-aligned wherein the bone wedge can be repositioned;

FIG. 14 shows a side view of a bone model wherein the bones are repositioned to correct for length;

FIG. 15 shows a lamina spreader inserted to spread the proximal and distal bones to correct for length;

FIG. 16 shows the reinsertion of the bone wedge after the re-alignment of the bone pieces along with an outline of a locking plate for securing the bone pieces;

FIG. 17 shows a compression screw that is used to fixate the proximate and distal shafts;

FIG. 18 shows excess callus on the lower halves of the proximal and distal bones that can be removed and used as a bone graft;

FIG. 19A shows the bones after fixation, but prior to the attachment of a compression plate;

FIG. 19B shows the compression plate positioned on top of the fixated bones and the possible attachment points for the compression plate;

FIG. 20A shows the proximal and distal bones prior to correction of the lesser angle of deformity;

FIG. 20B is a side view of the bone shown in FIG. 20A;

FIG. 20C is a perspective view of the bone of FIG. 20A with a surgical pin inserted into the bone.

FIG. 20D shows the proximal and distal bones after the correction of the lesser angle of deformity;

FIG. 21 shows a dynamic locking compression plate that can be attached to the bone pieces;

FIG. 21A shows an alternative compression plate that includes a lip;

FIG. 22 shows an attachment that can be attached to an embodiment of the jig for assisting in determining the central axes of the bone sections; and

FIG. 23 is side view of a blade for use with the jig that includes a set screw that provide a stop guide for the depth of the blade cut when used with the jig.

### DETAILED DESCRIPTION OF SPECIFIC EMBODIMENTS

Definitions. As used in this description and the accompanying claims, the following terms shall have the meanings indicated, unless the context otherwise requires: the term “deformity” shall mean some difference in a bone from a normal expected state that may arise either naturally or as a result of improper healing of a break or fracture.

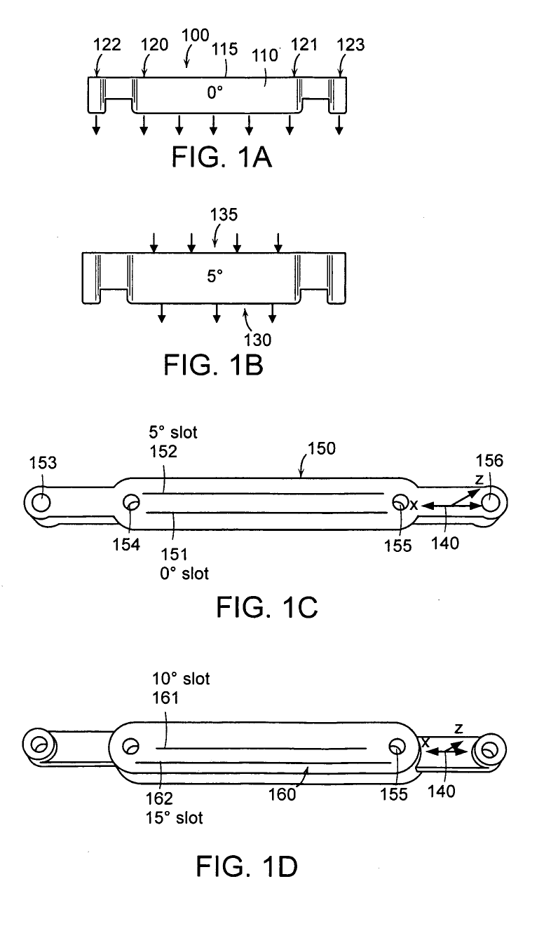

FIG. 1A shows a side view of a jig 100 for making a plurality of cuts creating an osteotomy in a bone that has an abnormality as to length and/or angle and/or rotation. The jig 100 includes a base 110 which has a slot 115 there through for guiding a blade along a path. The base 110 also includes a plurality of apertures 120, 121 for receiving a surgical pin and securing the base to the bone to be cut. In the embodiment of FIG. 1A there are four apertures, two 120, 121 that are within the base adjacent and on opposite sides of the slot and two 122, 123 that are at a fixed distance from the other two apertures. The jig 100 is coupled to the bone during surgery using one or more pins that can be inserted through the plurality of apertures.

FIG. 1B shows a side view of a jig 130 that is similar to the jig of FIG. 1A with the exception that the jig has a slot that is at five degrees relative to a longitudinal plane 140. The longitudinal plane passes through the body of the jig along the length of the jig. Thus, the blade when inserted through the slot will cut the bone at an angle relative to the longitudinal plane.

FIG. 1C shows a top view of an embodiment of the jig 150 wherein the jig has a plurality of slots 151, 152. As shown in the figure the first slot 151 is delineated as zero degrees and the second slot 152 is delineated as five degrees. The angle associated with both slots is relative to the longitudinal plane 140. The four apertures 153, 154, 155, 156 are visible and are sized to receive a guide pin. FIG. 1D show a top view of another embodiment of the jig 160 wherein the jig includes a plurality of slots 161, 162 at ten and fifteen degrees relative to the longitudinal plane 140. Each of the various angles from zero degrees from the longitudinal plane may be used to correct for rotational deformities as will be explained below.

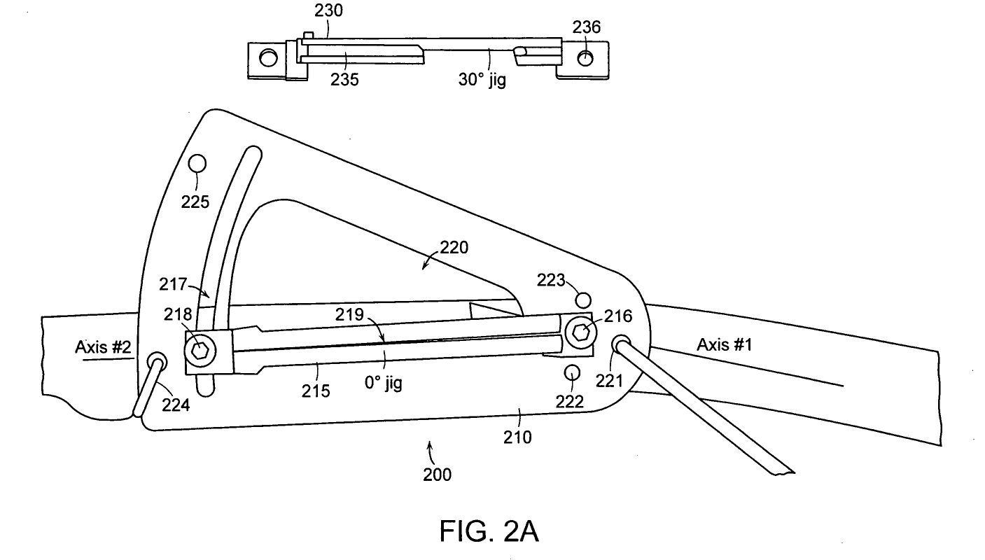

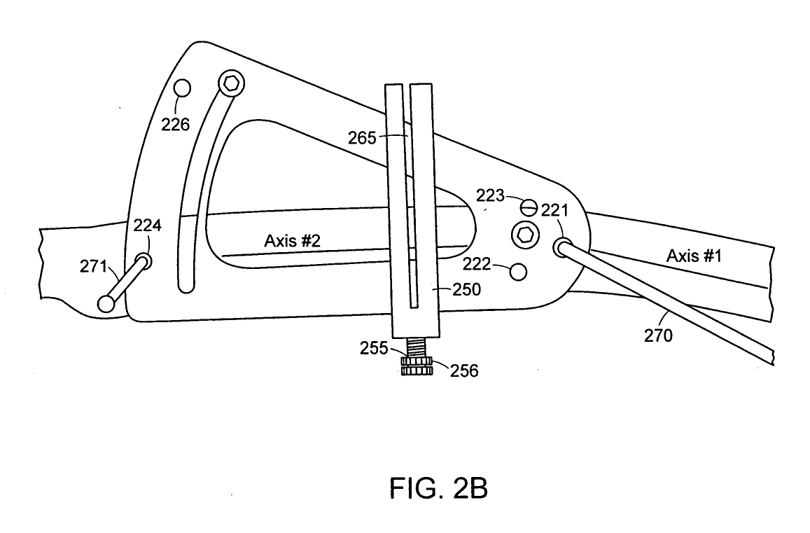

FIG. 2A shows a second embodiment of the jig 200, wherein the jig includes a base 210 and a pivoting slotted member 215. As shown, the base 210 of the jig 200 is substantially triangular in shape. The slotted pivoting member 215 is pivotally coupled at a pivot point 216 to the base at a first end point and resides in an arc-shaped void 217 in the base at a second end point. The slotted member includes a locking mechanism 218 that allows the pivoting member 215 to be tightened and secured at a fixed position within the arc-shaped void 217, so that the slot 219 can be used to guide a medical saw blade without the member pivoting 215. A second void 220 that is also substantially triangular in shape resides between the first and second end points of the slotted pivoting member 215. Much like the first embodiment of the jig as shown in FIGS. 1A-D, the base includes a plurality of apertures 221, 222, 223, 224, 225 for securing the base 210 to the bone during the osteotomy procedure. The pivot 216 that attaches the slotted pivoting member 215 to the base 210 is separably connectable. For example, the pivot 216 may be a bolt that passes through the base and the slotted member and is kept in position by a restraining nut. The nut may be loosened and the slotted member may be removed from the bolt. As shown in FIG. 2A. a second slotted pivoting member 230 is shown that has an indication that the slot 235 is at a 30 degree angle. The bolt may be passed through the pivot hole 236 of the 30 degree slotted pivoting member 230 and the nut may be tightened so that the 30 degree slotted member is attached to the base 210 and is allowed to pivot. The locking mechanism 218 that is used at the second end of the slotted member may similarly be composed of a nut and bolt wherein the nut may be loosened when the slotted member is to be pivoted.

FIG. 2B shows an embodiment of the jig of FIG. 2A wherein the pivoting slotted member is removed and a slotted planar member 250 is attached. The slotted planar member 250 can be attached to the base through a connection mechanism 255. As show, a screw 256 is screwed into the side of the base. The slotted planar member 250 allows a blade to be placed into the slot 265 and allows for a cut through the bone that is transverse to the slotted pivoting member's slot 219 as shown in FIG. 2A. As shown in the figure there are a plurality of apertures 221, 222, 223, 224, 225 through the base through which a pin 270, 271 may be placed for securing the base to the bone. Two pins 270, 271 are used to attach the base to the bone as shown in the Figure.

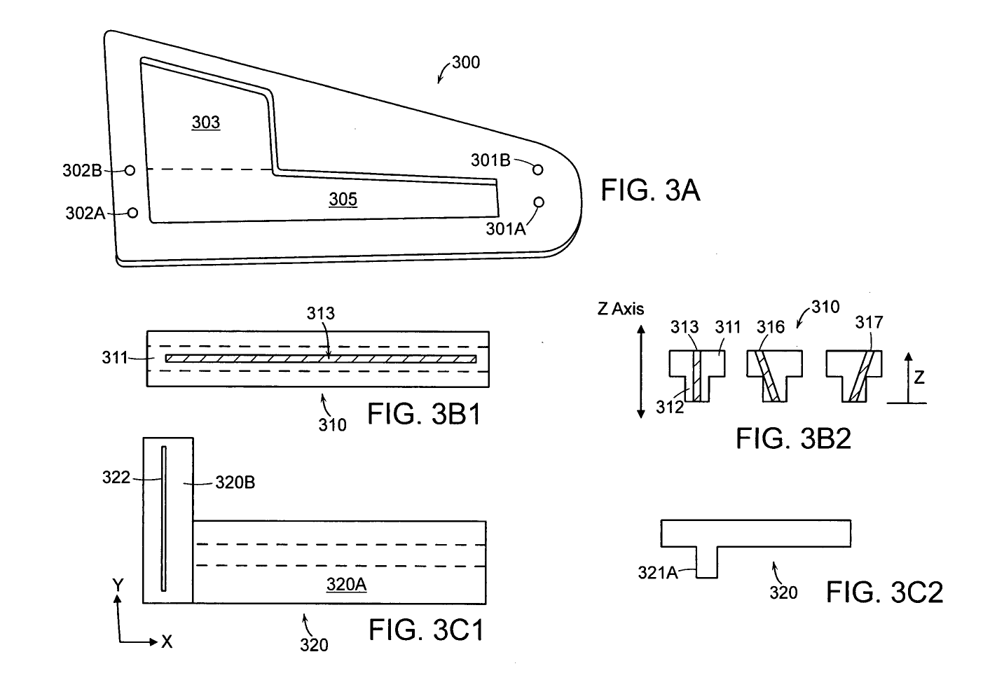

FIGS. 3A-3C2 shows the three components for a third embodiment of the jig. FIG. 3A shows a pivoting base 300 that pivots about a pin placed through one of the apertures 301A, 301B and inserted into the bone. The base also includes a second set of apertures 302A, 302B for securing the base 300 in place after it has been pivoted to the desired position. A void 303 in the base is formed for receiving the second and third components so that substantially perpendicular blade cuts may be made. FIG. 3B1 shows the second component of the jig 310 wherein the second component is T-shaped. The second component 310 is formed by an upper section 311 and a lower section 312, wherein the lower section fits longitudinally into the void of the first jig. The second component includes a slot 313 for receiving a saw blade there through. The slot 313 of the second jig 310 is narrow and more precisely formed as compared to the longitudinal section 305 of the void 303 of the first jig 300 and is sized to receive a saw blade.

The second component 310 as shown in FIG. 3B2 (end view) may include a plurality of slots wherein the saw blade may be placed there through and where each slot 313, 316, 317 has a different angle with respect to a Z-axis. For example, the second jig 310 may include slots at different angles. Three embodiments are shown in FIG. 3B2. The first embodiment shows the slot 313 substantially parallel to the Z-axis. The second embodiment of the second component shows the slot 316 thirty degrees off the Z-axis to the left. The third embodiment of the invention shows the slot 317 thirty degrees off the Z-axis to the right.

The third component 320 is shown in FIG. 3C1 and 3C2 from the top and end respectively. The third component 320 includes two sections. The first section of the third component is a longitudinal section 320A and the second section is a horizontal section 320B that is perpendicular to the longitudinal section 320A. The longitudinal section 320A is T-shaped and the shaft 321A of the T can be placed through the longitudinal section 305 of the void in the first component. The longitudinal section 320A is preferably shorter in length than the longitudinal section 305 of the void in the first component allowing the T-shaped shaft 321A of the longitudinal section 320A to be slid back and forth within the longitudinal void 305. The horizontal section 320B of the third component 320 includes a slot 322 for inserting a saw blade. The horizontal section 320B is substantially parallel to the Y-axis. Thus, the slots 313, 322 from the second component 310 and the third component 320 are transverse and substantially perpendicular.

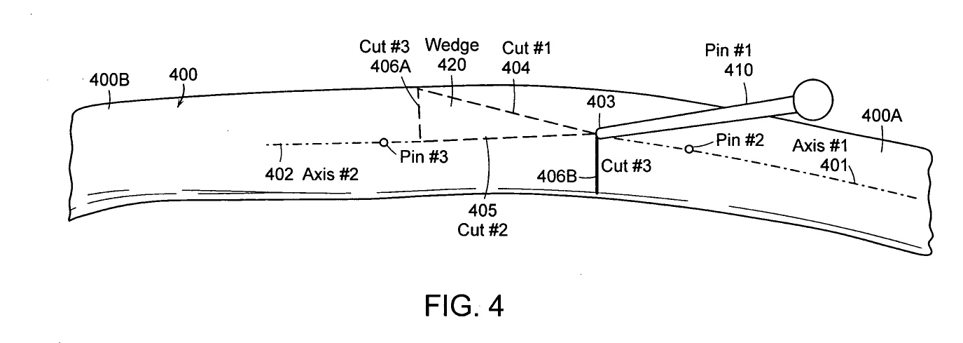

The methodology for using the previously described embodiments of the jig will now be presented. The methodology will be explained with respect to the jig as embodied in FIGS. 1A-D; however different embodiments of the jig may be used to perform the same procedure for creating an osteotomy and correcting for length, angular and rotational deformities. FIG. 4 is a side view of a bone 400 having an angular deformity. The center of the deformity is first defined as separated between a first bone section 400A and a second bone section 400B. A first central axis 401 is defined on a first section of the bone 400A. This axis can be physically defined by marking the bone or defined on a real-time imaging device such as a fluoroscope. Additionally, the markings can be made using an x-ray image of the bone, wherein the x-ray image is aligned with the actual bone and the axis can then be marked on the bone. A second axis 402 is then defined along the second section of bone 400B in a similar fashion as the first axis. The first and second axes 401, 402 meet at a point 403 which is the center of the deformity. A medical guide pin 410 is used to identify this location and the pin 410 is later used in conjunction with the jig as a pivot point. The pin 410 is drilled into the bone at a right angle to the bone.

Cuts are made so as to form a bone wedge 420 about the deformity point. A first cut 404 is made along the first axis 401 on the second section of the bone 400B forming the hypotenuse of a triangle. The cut is made from the deformity center 403 to the edge of the bone cortex. A second cut 405 is made on the second axis 402 on the second section of the bone 400B. This second cut 405 forms the base leg of the triangle and is preferably aligned with the point of the first cut that is at the cortex edge of the bone. A third cut 406A is made substantially perpendicular to the second cut 405 so as to create a bone wedge 420 that can be removed. This cut 406A is a convexity cut. The osteotomy is complete with a cut 406B that is substantially parallel to the third cut which is between the concavity and the central deformity point. The bone 400 is then separable into a first and second section 400A, 400B. The foregoing cuts are capable of correcting for both length and angular deformities.

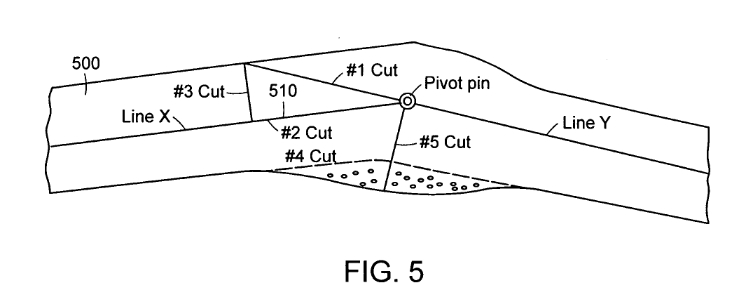

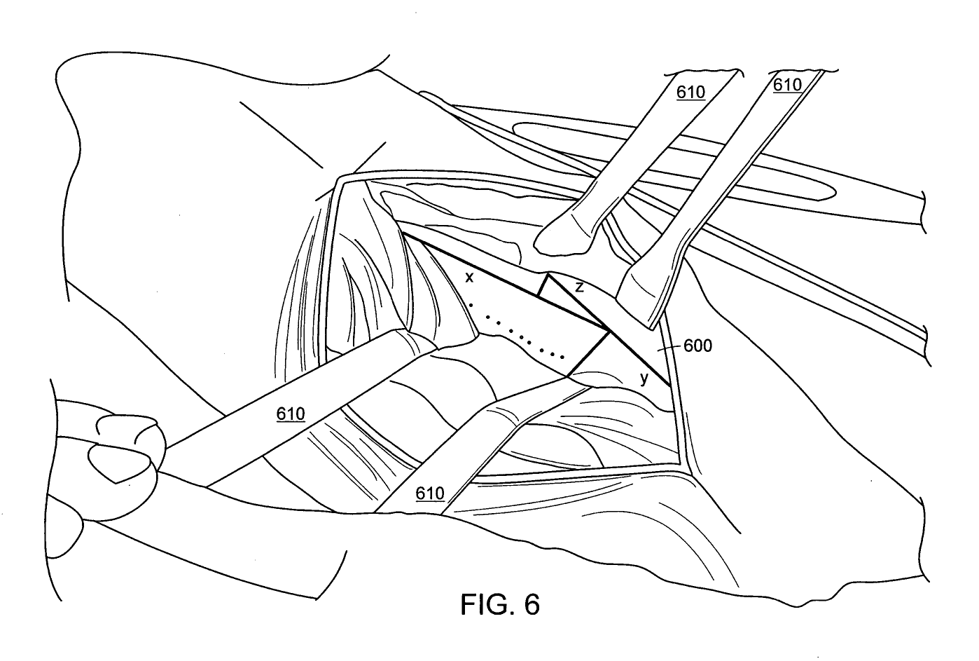

FIG. 5 is a side view of a bone 500 having a deformity along with the denoted cut lines including a cut line for rotational correction. A rotational correction can be made by using an angled cut 510 along the X axis at the same location as the second cut as described above with respect to FIG. 4.

FIG. 6 is a photograph of a radius 600 mal-union that is short, angulated and rotated. First an incision is made proximate to the central location of the deformity. Spreaders 610 are used to pull the muscle away from the bone 600 so that the bone 600 is exposed. As shown in the figure, the axes of FIG. 4 and 5 can be drawn onto the bone and the central point of the deformity is defined where the x and y axes cross.

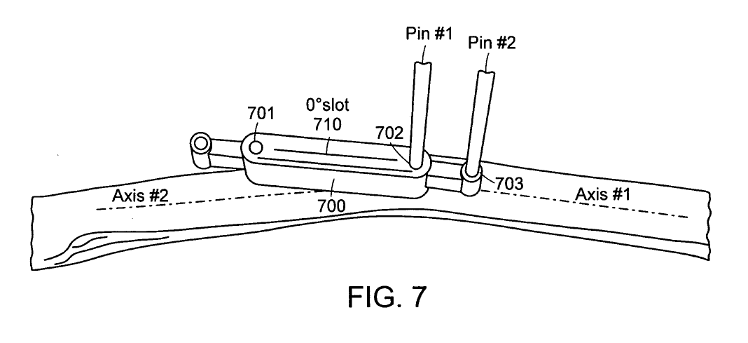

The use of the jig will now be described in the context of the cuts that create the osteotomy for realignment and correction of the bone deformity. FIG. 7 shows a first position of the jig of FIGS. 1A-D. A zero degree guide as shown in FIG. 1A is used. After the axes have been defined (axis 1 and 2 as shown) and the medical pin (pin #1) is inserted into the bone at the central deformity, the jig is placed over the guide pin (pin #1) through one of the two central apertures 701, 702 as the jig is symmetrical. The jig 700 is then aligned with axis 1 and a second guide pin (pin #2) is drilled into the bone through a second aperture 703 that lies along axis 1. This second pin (pin #2) secures the jig 700 in place so that the jig does not rotate during use of the drill (not shown) when the first cut is made. A saw blade is then used through a zero degree slot 710 in the jig wherein the cut is from the cortex to a point proximate to the central point where pin #1 is located. Preferably, the saw blade's thickness is such that it tightly fits into the slot, allowing for a precise cut. The blade may also include an adjustable stop, so that the depth of the cut can be determined. The adjustable stop can be a series of screw holes on the blade where a screw is inserted there through at different positions for different blade depths. The adjustable stop allows the same size blade to be used with patients of different sizes or for the blade to be used on different bone types. Blade depth markings are provided on the blade next to the screw holes for the adjustable stop, and account for the height of the jig, so as to provide the surgeon with an accurate measurement of depth. It should be understood, that a blade stop may be included with any of the blades that are used with any of the disclosed embodiments of the invention.

In this embodiment of the jig 700, the cut does not reach the pivot point (central location of the deformity). In other embodiments, the slot may extend to the pivot point. The reason for the slot not extending to the pivot point in the present embodiment is to provide bone integrity during the second and third cuts, wherein the wedge will be removable after the second and third cuts are made with a final manual sawing of the bone to the pivot point.

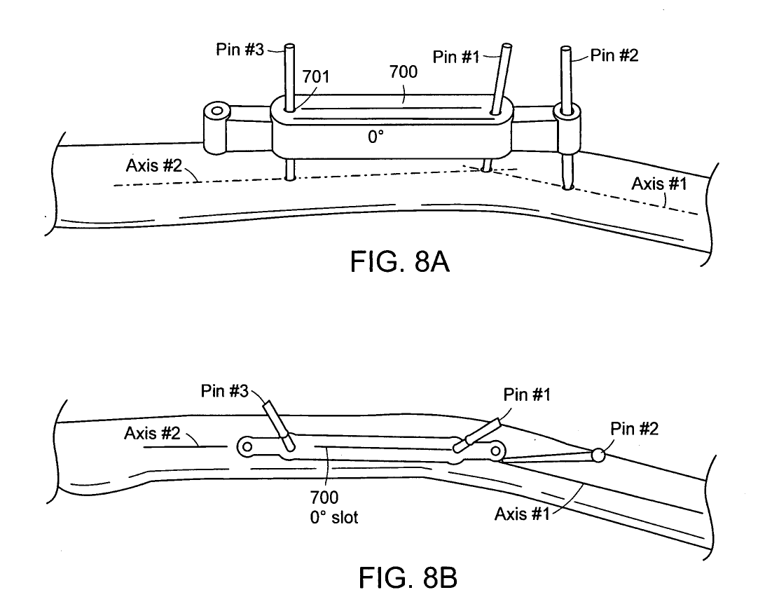

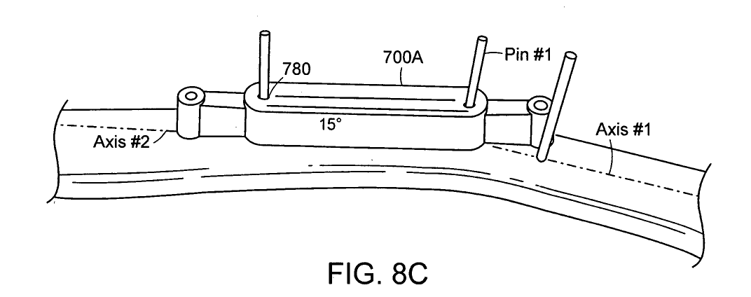

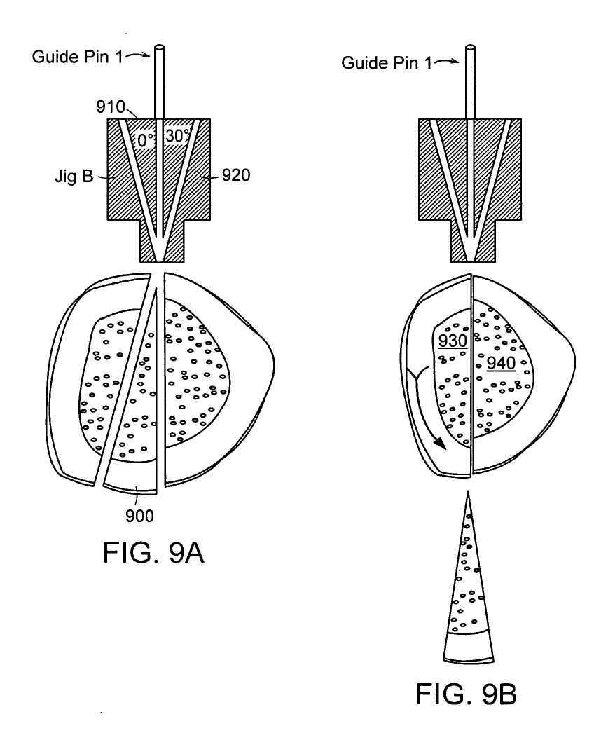

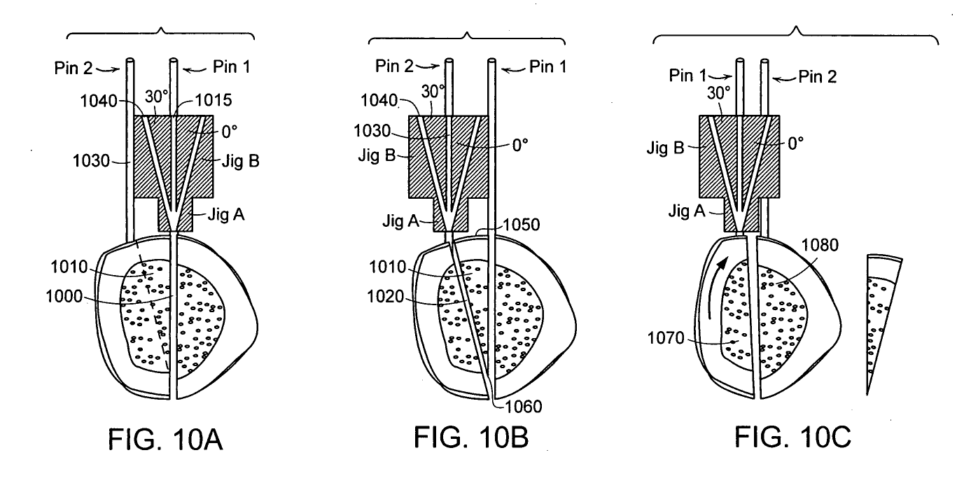

FIG. 8A shows a perspective view of a second position of the jig 700. The jig is removed from both the first and second pins and the jig is reinserted on pin #1 and is aligned with Axis #2 as shown. Pin #2 may be left in place or removed. Once aligned, a third pin (pin #3) is inserted through a third aperture 701 to secure the jig in place during the second cut. This third aperture 701 may be located on the opposite side of the slot from the pivot point defined by pin #1. FIG. 8B shows a top view of the second position of the jig 700. In FIGS. 8A and 8B, it is assumed that there is no rotational deformity of the bone. If a rotational deformity is present in the bone, a jig 700A with an angled slot is subsequently used as shown in FIG. 8C. After the second cut is made using the jig 700 as positioned in FIG. 8A and 8B, the jig is either removed and a new jig 700A that has an angled slot is used or a different slot on the same jig examples of which are shown in FIGS. 1B-D. The jig is placed on pin #1 and attached through a third aperture 780 by pin #3 on axis #2 as defined in the figure. By using the jig 700A that has a slot at an angle (ex. 15 degrees as shown) and using a saw blade through the slot across axis #2, a wedge shape (not shown) is formed wherein the base of the wedge is on the opposite side of the bone from the jig. The two halves of the bone can then be rotated after the wedge is removed and subsequent to the third and fourth cuts which complete the osteotomy. Rotational correction in a counter-clockwise and clockwise manner are further explained in FIGS. 9 and 10 respectively.

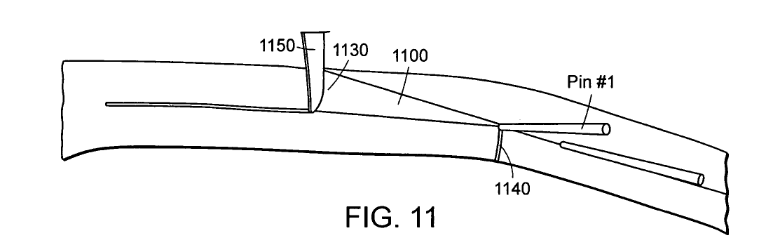

FIGS. 9A-B show a cross-sectional view and correction of rotation of a bone in a counter-clockwise rotational direction. FIG. 9A shows the wedge 900 that is formed after the two rotational cuts are made in the bone. The first rotational cut is cut number 2 that is made along axis #2 as shown above in FIGS. 8A-B. The blade is placed through a zero degree slot 910 that cuts through the bone. Then a second rotational cut is made on the same axis by inserting the blade through the 30 degree slot 920 in the jig as shown. The wedge shape 900 can then be removed. The amount of rotational correction can be varied depending on the angle of the slot. Using a zero degree and a 30 degree slot, the bone is corrected for 30 degrees of rotation. The amount of rotation can be determined by the linea. The linea, which are the ridges in the bone should align. If the crests of the linea do not align, rotational correction is appropriate. FIG. 9B shows the re-assembled bone pieces 930, 940 with an arrow indicating the counter-clockwise rotation of one of the bone pieces. Rotation of the bone pieces can only be performed after the osteotomy is completed even though the rotational cuts are made. As taught in one embodiment, the osteotomy is completed by the third and fourth cuts to the bone as are explained below with respect to FIG. 11.

FIGS. 10A-C are cross-sectional views of a bone wherein the bone is being corrected in a clockwise rotational direction. FIG. 10A shows a first cut 1000 of the bone to form the rotational wedge 1010. The first cut 1000 is performed as shown in FIG. 8A and 8B wherein the slot 1015 of the jig is at a zero degree angle with respect to axis #2. The cut 1000 is made through the entire bone as shown. FIG. 10B shows a second cut 1020 that forms the bone wedge 1010. The jig is moved by several millimeters parallel to axis #2 (this distance can be calculated from the geometry, including the desired angular rotational correction and the diameter of the bone). The jig 1030 is secured and a 30 degree slot 1040 is used to make the cut 1020. Thus, the base of the wedge 1050 is formed at the jig and the point 1060 is at the other side of the bone. FIG. 10C shows the two bone pieces 1070, 1080 realigned after the bone wedge has been removed. The left most bone section 1070 is rotated the 30 degrees for a 30 degree correction.

If the surgeon needs only to make a rotational correction to the bone and does not need to correct for angular defects, the first and second cuts are equivalent, as the axes of the bone sections are in alignment (Axis #1 and #2 align). Thus, the surgeon would make a first cut along center axis of the bone, a second cut substantially perpendicular to the first cut at a first end of the first cut and a second cut at the second end of the first cut that is substantially perpendicular to the first cut. Thus, an osteotomy of the bone is created. Additionally, an angled cut can be made along the first cut, in order to create a bone wedge that can be removed. Once the bone wedge is removed, the two bone pieces can be rotated as shown in FIGS. 9 and 10.

FIG. 11 shows the positions of the third and fourth cuts that 1130, 1140 complete the osteotomy and create a removable bone wedge 1100 for correction of an angular or lengthwise deformity (cuts 1 and 2 are shown in FIGS. 7 and 8A, 8B the cuts that are made for rotation are only made if necessary). The third cut 1130 can be made using a jig, straight edge or can be made freehand. This third cut 1130 is made perpendicular to axis #2 starting at the cortex of the bone at the point where the first cut came through the bone cortex and ending at axis #2. This third cut 1130 completes the bone wedge. The blade 1150 in the Fig. is shown making this cut. The fourth cut 1140 is made perpendicular to pin #1 and completes the osteotomy so that the bone is in two separable pieces.

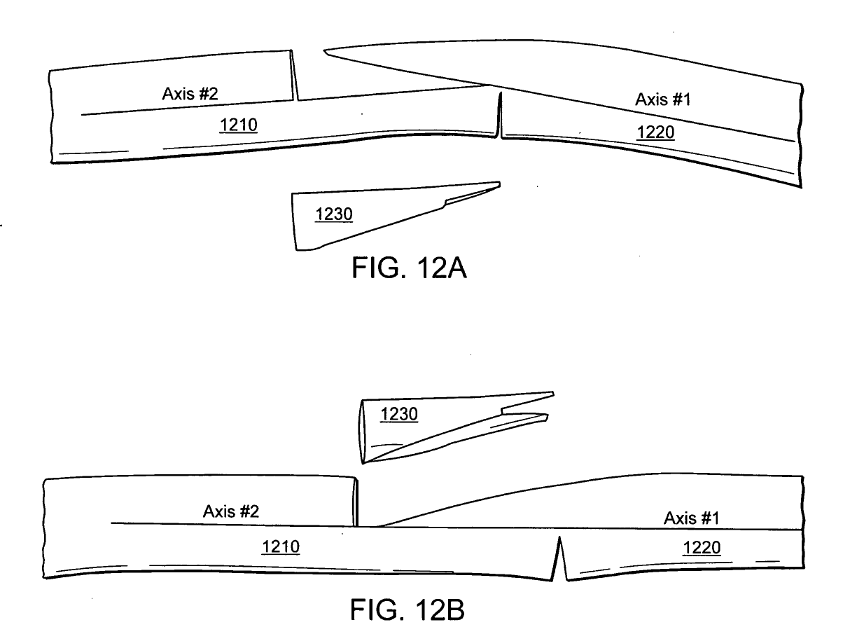

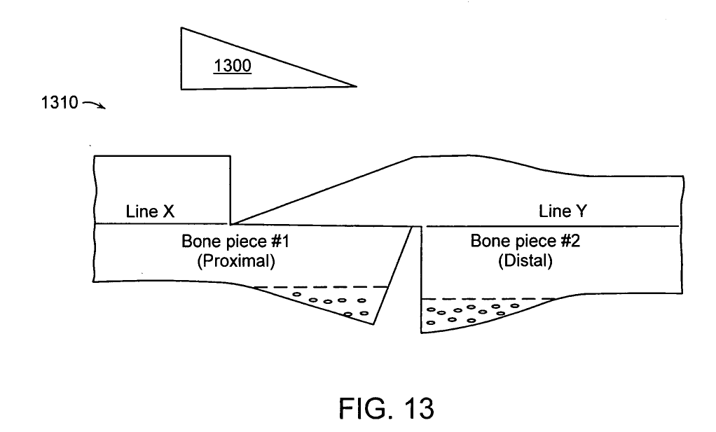

FIG. 12A shows the two bone pieces 1210, 1220 that are formed during the osteotomy immediately after the bone wedge 1230 is removed. FIG. 12B shows the two bone pieces 1210, 1220 after the center axes (axis #1, axis #2) have been re-aligned. Thus the greater angle of deformity is corrected having the central axes aligned.

FIG. 13 shows another view of the two axes re-aligned (line X, line Y) wherein the removed bone wedge 1300 can be repositioned. Due to the geometry of the cuts, the bone wedge 1300 is sized to readily fit into the larger of the two created gaps 1310 that occur due to re-alignment of the bone pieces. After correcting the greater angle of deformity, the bone pieces can be rotated if rotational correction is necessary as explained above with respect to FIGS. 8C, 9A,B and 10A,B,C.

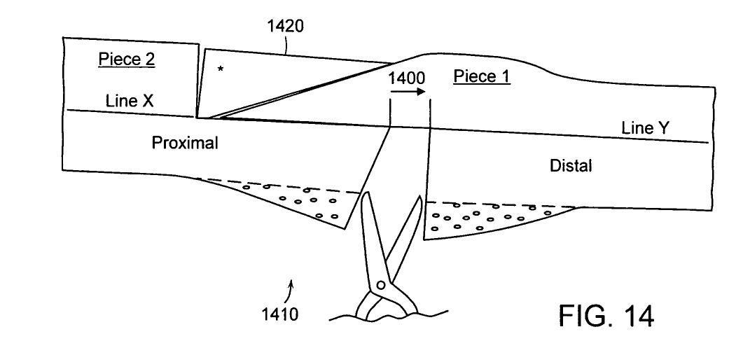

After realignment of axis X and axis Y, the bone pieces 1 (proximal) and 2 (distal) can be moved with respect to one another to correct for length with a lamina spreader 1410 as shown in FIG. 14. The lamina spread 1410 is opened and the bones are distracted about 1400. FIG. 14 also shows the reinsertion of the bone wedge 1420 that had previously been removed during the osteotomy procedure. The bone wedge 1420 need not be reinserted during distraction of the bones.

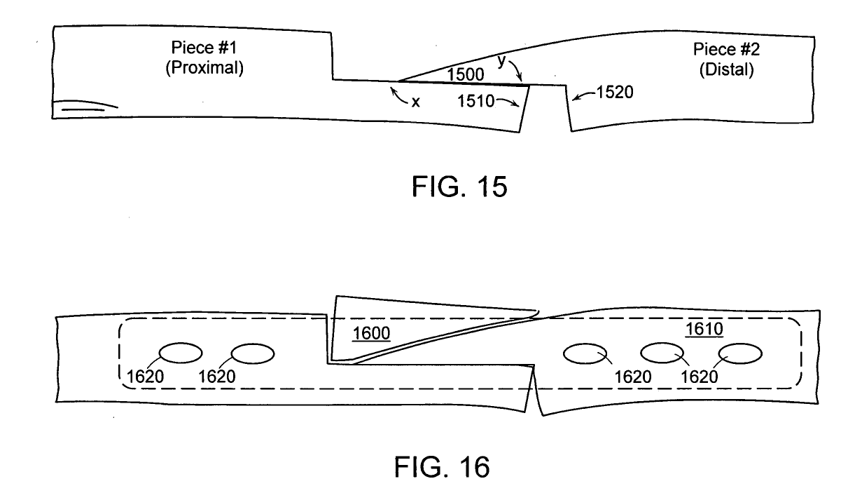

FIG. 15 shows a side view of a bone model after the lamina spreader has been used. Contact between the bone pieces is maintained along surfaces X and Y during the distraction. The methodology also allows for the bone to be shortened. Shortening of the bone can be achieved by compressing the bones and removing a portion of bone from either the proximal or distal piece. For example, the tip 1500 of the distal bone piece may be removed or bone may be shaved or cut off the bone surfaces 1510 and 1520 of the proximal and distal bones pieces respectively.

FIG. 16 shows the reinsertion of the bone wedge 1600 as a bone graft between the proximal and distal bone pieces after realignment. Once the bone wedge 1600 is inserted, a locking compression plate 1610 is placed over the bone pieces which secures the bone pieces together. As shown the plate 1610 has a number of apertures 1620 for receiving screws.

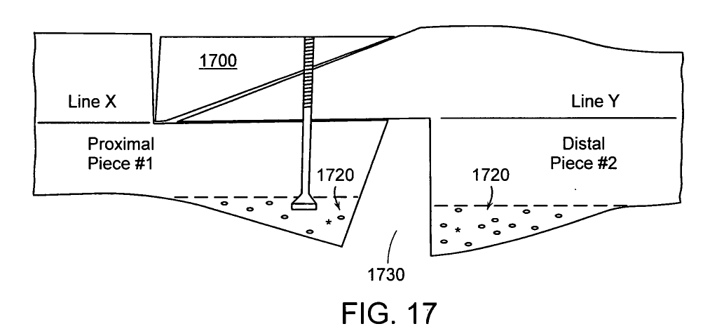

FIG. 17 shows a compression screw 1710 that is used to fixate the proximate and distal bone pieces. As shown, the bone pieces are in alignment and the compression screw 1710 is drilled through the bone, so that the screw 1710 contacts at least the distal and proximal bone pieces. The screw fixates the pieces so that the bone pieces cannot rotate. In the Fig., the compression screw 1710 also contacts the bone wedge graft 1700 and holds the graft in place relative to the proximal and distal pieces of the bone.

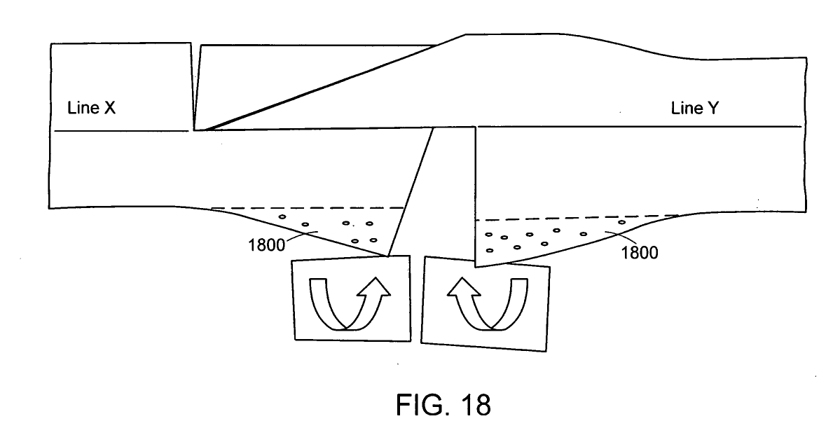

As shown in the figure, there is excess callus 1720 from the bone on the lower part of the distal and proximal bone pieces. If this callus 1720 is present on the bone of a patient, the callus 1720 can be removed by shaving off the callus. The bone callus 1720 can then be used as an additional source of bone graft and may be added to the gap 1730 on the concave side of the corrected bone. If excess bone is not available, then allograft, autograft, or synthetic graft can be used. FIG. 18 shows excess callus 1800 on the lower halves of the proximal and distal bone segments that can be removed and used as a bone graft.

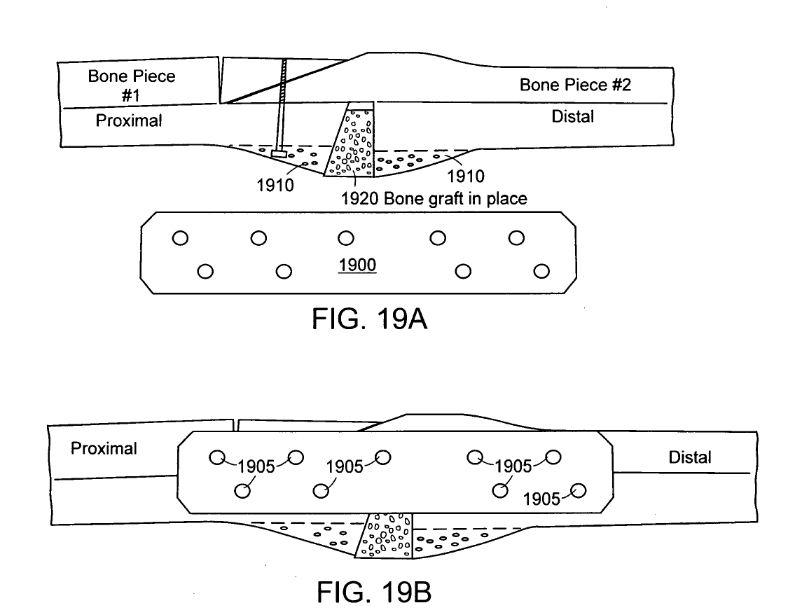

FIG. 19A shows a top view of the bone pieces after fixation, but prior to the attachment of the compression plate 1900. The callus 1910 has not been removed from this bone, however a bone graft 1920 has been added to fill in the gap on the lower portion of the bone. This Fig. shows the correction of the greater angle of deformity.

FIG. 19B shows the compression plate 1900 positioned on top of the fixated bones and the possible apertures for attachment of the compression plate 1905. The dynamic compression plate 1900 includes a plurality of apertures 1905 for fixation of both the proximal bone section and the distal bone pieces. The plate 1900 may also include one or more openings for insertion of a screw for stabilizing one or more of the bone wedges.

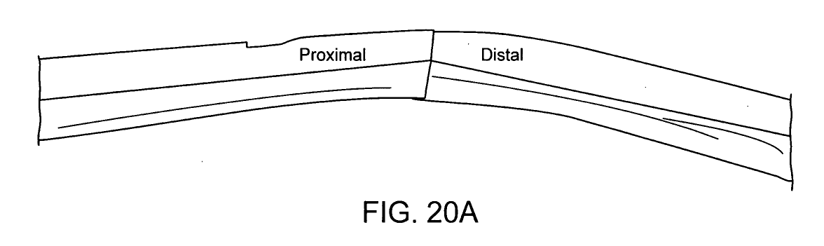

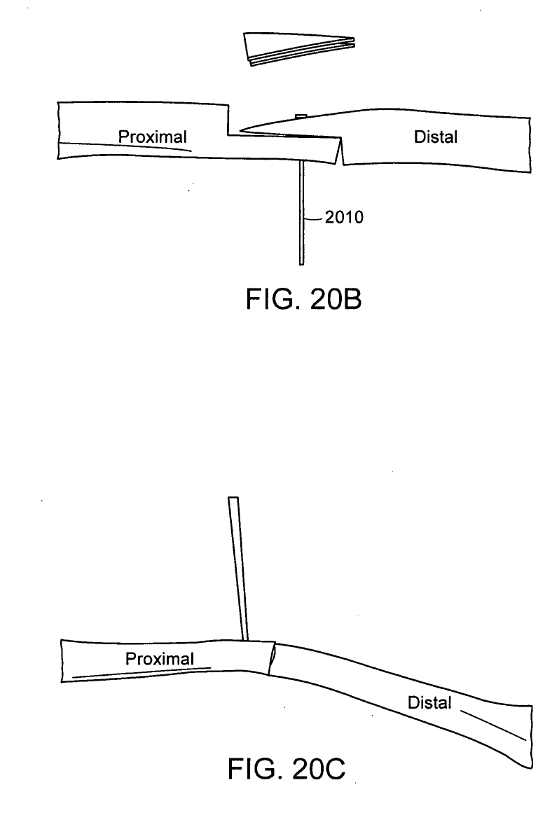

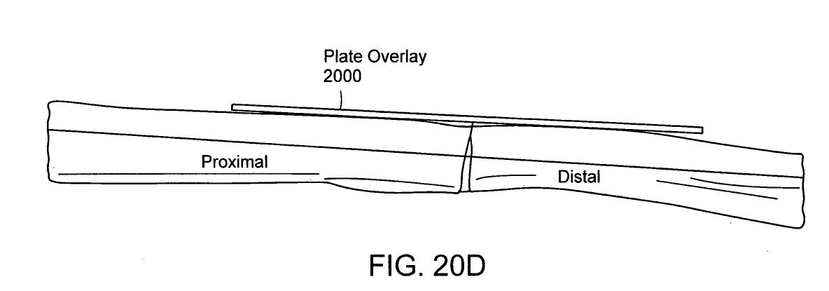

A lesser angle of deformity may be corrected after the osteotomy is complete and the greater angle of deformity has been corrected by aligning the central axes of the proximal and distal bone sections. The lesser angle of deformity is in a plane that is perpendicular to the greater angle of deformity. FIG. 20A is a top view of a bone that shows the proximal and distal bones pieces prior to correction of the lesser angle of deformity. The proximal and distal bone sections are not in alignment and there is an angular deformity. FIG. 20B is a side view of the bone. The central axes of the proximal and distal bone sections have been realigned correcting the greater angle of deformity as previously described. A surgical pin 2010 is driven through both the proximal and distal bone pieces perpendicular to the central axes. The pin 2010 provides a pivot point for correcting the angle of lesser deformity. FIG. 20C shows a perspective view of the bone showing the surgical pin 2010 that is perpendicular to the plane of the angle of lesser deformity. The pin 2010 from FIG. 20C is shown. Thus, the proximal and distal bone pieces may be rotated about the pin 2010 and the lesser angle of deformity can be reduced. FIG. 20D shows the proximal and distal bones after the correction of the lesser angle of deformity with the surgical pin removed. For reference, a side view of the compression plate 2000 is shown on the upper surface of the bone.

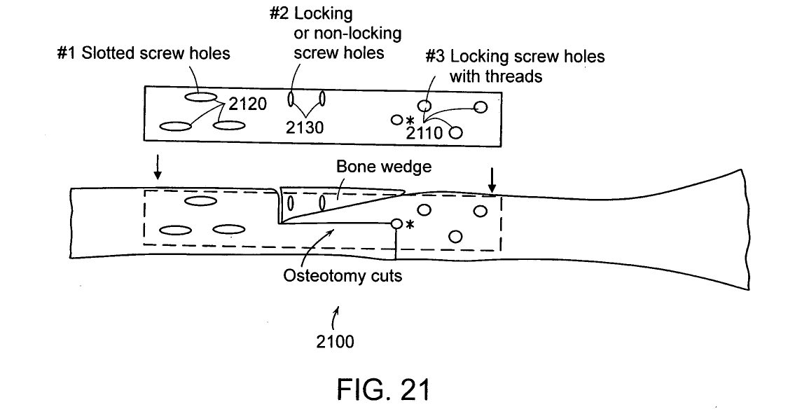

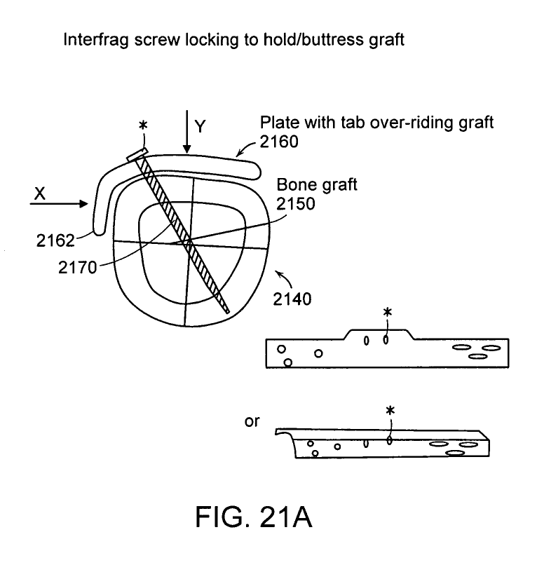

FIG. 21 shows an embodiment of the compression plate 2100. The compression 2100 may be constructed from stainless steal or titanium or another durable material. The embodiment of the compression plate 2100 that is shown includes a guide pin hole. The guide pin hole is marked with an asterisk in the Fig. The guide pin (not shown) that is inserted at the center of the deformity is aligned with the guide pin hole of the compression plate and the plate is placed over the pin and onto the bone sections. This allows the plate 2100 to be positioned so that the plate 2100 can be securely coupled to each bone piece. The plate includes a plurality of screw holes for receiving cortex or cancelleous bone screws. On the right side of the plate are three holes 2110 that are for ordinary locking bone screws. Locking screws provide the advantage that the screws will not slide or become dislodged from the bone over extended use. On the left side of the compression plate are elongated slots 2120. The walls of the slots are tapered wherein the walls at the bottom side of the plate that is against the bone pieces is narrower than the walls at the top side of the plate. The screws that are used with the elongated slots include a head that is tapered in a similar fashion. The tapering of the walls of the slot allows the plate to provide compression or distraction depending upon which side of the slot that the screw is placed. If the screw is placed in the slot toward the left side of the slot as shown in the Fig. and screwed in place, the screw will be pushed to the right as it is drilled into the bone. This will slightly move the bone to the right and create distraction. If the screws are placed into the slot on the right side of the slot, the screws will be pushed to the left moving the bone piece slightly to the left and creating compression between the bone pieces.

Additionally, the plate may include a slot or hole for a screw 2130 that is positioned on the plate 2100 under which the wedge shaped bone graft is to be located. A screw can then be screwed into the bone graft securing the bone graft to the plate.

FIG. 21A shows a cross-sectional view of a bone 2140 on which an osteotomy has been performed. An alternative version of the compression plate 2160 is used to secure the bone pieces together. This version of the compression plate 2160 includes an extended lip 2162 and is L-shaped. The extended lip 2162 provides support for the bone graft 2150 in two directions (X and Y) so that the bone graft 2150 will not slip out of place over time. The compression plate 2160 also includes a threaded slot for receiving a locking interfragmentary screw 2170. The interfragmentary screw 2170 secures the bone graft 2150. If locking screws are used, the screws may buttress the graft in addition to fixation.

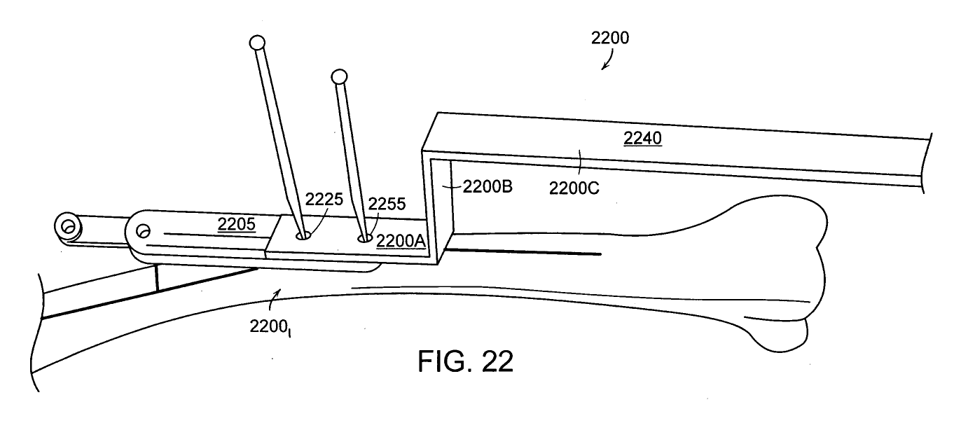

FIG. 22 shows an attachment 2200 that has been attached to the jig 2205 for assisting in alignment of the axes prior to making the cuts. This attachment 2200 allows the axes to be determined after a surgeon inserts a pin by “free handing” the central position of the deformity of the bone. As stated the surgeon first free hands the location of the central position of the deformity and inserts a pin 2210 into the bone at that location. The jig 2205 is then placed over the pin 2210 and the jig 2205 rests on the bone 2220. The attachment includes an aperture 2225 and the guide pin 2210 is placed through the aperture 2225. The attachment 2200 attaches to the jig 2205 using an attachment means such as a plurality of tabs 2230 securely engage the body of the jig 2205. As shown, the attachment 2200 has three sections. The first section 2200A engages the jig, includes the locking means 2230, and includes the aperture 2225. This first section 2200A has a central axis that is aligned with the central slot of the jig. The second section 2200B is perpendicular to the first section 2200A. The third section 2200C is perpendicular to the second section 2200B. The third section 2200C includes a marker 2240 that resides along the central axis of the third section 2200C. The marker 2240 is capable of being fluoresced and will show up on a fluoroscope when imaged. Once the attachment 2200 is properly positioned, a real-time fluoroscope is used to image the bone. The marker is aligned by the surgeon to the center axis of the bone by rotating the attachment 2200 and the coupled jig 2205. Once the marker 2240 is properly aligned, a second pin 2250 can be inserted through an aperture 2255 in the jig 2205 and the attachment 2200. Once this first central axis of the bone is defined, the axis can be marked on the bone 2220. The jig 2205 and attachment 2200 can then be used to locate the central axis of the second section of the bone. Once the two axes are located and marked, the attachment 2200 can be removed from the jig 2205, and the jig 2205 can be used to make the cuts as previously described depending on the deformity that is being corrected for.

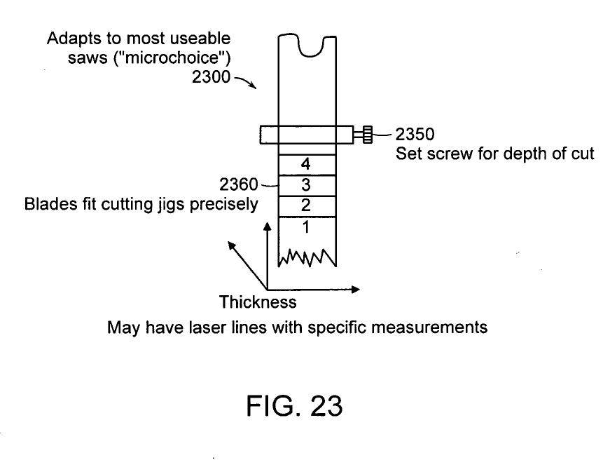

FIG. 23 shows a partial side view of a blade 2300 for use with the previously described jigs. The blade 2300 has a thickness that is sized to fit into the slot of the jig. The thickness of the blade 2300 is such that the blade can not move or shift along the axis of the blade's thickness while the blade is in the slot and cutting a bone. The close tolerance between the blade's thickness and the slot's width allows the blade to make precision bone cuts. The blade 2300 may also include a set screw 2350 that allows for changes to the depth of the bone cuts. Along with the set screw is a plurality of measurement lines 2360. The measurement lines are preferably etched into the blade and allow the surgeon to align the stop with the desired depth of the cut. The blade depth measurements are such that they account for the thickness of the jig. When the set screw is put in place and the blade is inserted into the slot on the jig, the set screw will prevent the blade for extending further into the slot and therefore further into the bone.

This technique with the attachment and fluoroscope can be used to either determine the axes initially or to confirm the central location of the axes that have already been marked on the bone.

Although various exemplary embodiments of the invention have been disclosed, it should be apparent to those skilled in the art that various changes and modifications can be made that will achieve some of the advantages of the invention without departing from the true scope of the invention. These and other obvious modifications are intended to be covered by the appended claims.

1. An osteotomy device for determining osteotomy cuts for a deformity in a bone, the osteotomy device comprising:
a first jig having a first end and a second end with a longitudinal slot substantially parallel to an X-axis between the first end and the second end, the first jig also including a cutout wherein at least one edge of the cutout is substantially perpendicular to the longitudinal slot, the first jig further having a pivot point for receiving a pin for rotatably mounting the first jig to the bone.

2. The osteotomy device of claim 1, further comprising:
a second jig adapted to fit within the longitudinal slot of the first jig and having a slot configured to receive a saw blade.

3. The osteotomy device of claim 1, further comprising:
a third jig having a first section and a perpendicular second section substantially parallel to a Y-axis, the first section configured to fit within the longitudinal slot of the first jig and the second section having a slot configured to fit a saw blade.

4. A method for correcting a deformity of a bone wherein the bone has a first section and a second section that meet at a central point in the deformity, the method comprising:
defining a first axis along the center of the first section of the bone;
defining a second axis along the center of second section of the bone that intersects with the first axis;
placing a first pin securing a jig at the intersection of the first and second axes;
making a first cut along the first axis in the second section of bone using the jig to guide a blade;
making a second cut along the second axis in the second section of bone using the jig to guide the blade;
making a third cut transverse to the second cut such that a substantially triangular wedge of bone may be removed; and
forming a forth cut that fully separates the first section and the second section of the bone;
removing the substantially triangular wedge of bone; and
positioning the first section and the second section such that the first and second axes are substantially aligned.

5. The method according to claim 4, further comprising:
using the bone wedge as a bone graft to fill any voids left with the bone upon realignment of the first and second sections.

6. The method according to claim 4, attaching a plate to the first and second sections.

7. The method according to claim 6, wherein the plate is a locking plate.

8. The method according to claim 6, wherein the plate is a non-locking plate.

9. The method according to claim 4, wherein the second cut is made using a jig having a slot that is at an angle with respect to a plane that includes the first pin.

10. The method according to claim 4, wherein a second pin is inserted into the bone to secure the jig prior to making the first cut.

11. The method according to claim 4, wherein the jig is rotated about the first pin between making the first and second cuts.

12. The method according to claim 10, wherein a third pin is inserted into the bone to secure the jig prior to making the second cut.

13. The method according to claim 9, wherein the second cut is made completely through the bone longitudinally further comprising:
positioning a jig to make a cut that is parallel to the second cut completely through the bone longitudinally, so as to split the bone into three longitudinal sections wherein a rotational bone wedge is created;

14. The method according to claim 13, further comprising:
removing the bone wedge from the bone;
rotating one of the remaining two longitudinal section of the bone so as to correct for a rotational deformity in the bone.

15. A guide for use in correcting a deformity of a bone, the guide comprising:
a body having first end and a second end and a slot between the first and second ends, the slot for receiving a blade for cutting the bone wherein the slot has a proximal end proximate to the first end of the body and a distal end that is proximate to the second end of the body;
wherein the body has a plurality of apertures for receiving guide pins there through for securing the body to the bone, wherein at least one aperture is present between the first end of the body and the proximal end of the slot.

16. The guide according to claim 15, wherein at least one aperture is present between the second end of the body and the distal end of the slot.

17. The guide according to claim 15, wherein a plurality of apertures are present between the first end of the body and the proximal end of the slot.

18. A guide according to claim 15, wherein the body includes three or more apertures for receiving guide pins.

19. A guide for use in correcting a deformity of a bone according to claim 15, wherein an axis extends through the body and the slot cuts through the axis at an angle with respect to a plane that includes the axis.

20. A guide according to claim 19, wherein the body includes a plurality of slots that are at different angles with respect to the axis.

21. A method for correcting a deformity of a bone wherein the bone has a first section and a second section that meet at a central location of the deformity, the method comprising:
using a jig to make a plurality of cuts proximate to the deformity creating an osteotomy;
removing a substantially triangular piece of the bone formed by the plurality of cuts; and
aligning the first section and the second section of the bones.

22. The method according to claim 21, further comprising:
attaching the first and second sections of the bone to a plate.

23. The method according to claim 21 further comprising:
using the substantially triangular piece of the bone as a bone graft between the first and second sections of the bone.

24. The method according to claim 21 wherein the jig includes a slot for guiding a saw blade.

25. The method according to claim 21 further comprising:
determining a first central axis on the first section of the bone;
determining a second central axis on the second section of the bone that crosses the first axis at a point;
attaching the jig to the bone at the point.

26. The method according to claim 21 wherein making a plurality of cuts includes:
making a first cut along the first axis in the second section of bone using the jig to guide a blade; and
making a second cut along the second axis in the second section of bone using the jig to guide the blade.

27. The method according to claim 26, wherein making a plurality of cuts includes:
making a third cut transverse to the second cut such that the substantially triangular wedge of bone may be removed.

28. The method according to claim 27, further comprising:
forming a forth cut that separates the first section and the second section of the bone.

29. A blade for use with the guide of claim 15 wherein the slot in the base has a thickness and wherein the blade has a thickness substantially equal to the slot.

30. The blade according to claim 29, further including demarcations as to the depth of the cut, wherein the demarcations account for a thickness of the base of the guide.

31. A blade for use with the guide of claim 15 further including a blade stop for stopping the blade from inserting further into the slot.

32. The blade according to claim 31 wherein the blade includes a plurality of apertures and the blade stop is removably connected to the blade, such that depth of cut into the bone can be adjusted.

33. The blade according to claim 31 wherein the blade includes a plurality of etched lines with indicia as to depth.

34. The blade according to claim 31 wherein the blade has a thickness that is substantially equivalent to a width of the slot of the jig for forming a precision bone cut.
